(19) United States
(12) Patent Application Publication
(10) Pub. No.: US 2025/0312271 A1
(43) Pub. Date: Oct. 9, 2025

(54) GSK-3-BETA INHIBITOR-LOADED POLYMERIC SCAFFOLDS FOR THE TREATMENT OF MUSCLE INJURIES

(71) Applicant: Rutgers, The State University of New Jersey, New Brunswick, NJ (US)

(72) Inventors: Doga Oruc, New Brunswick, NJ (US); Joseph W. Freeman, Piscataway, NJ (US)

(73) Assignee: Rutgers, The State University of New Jersey, New Brunswick, NJ (US)

(21) Appl. No.: 19/169,158

(22) Filed: Apr. 3, 2025

Related U.S. Application Data
(60) Provisional application No. 63/574,120, filed on Apr. 3, 2024.

(51) Int. Cl. A61K 9/00; A61K 31/506; A61K 47/34; A61K 47/42; A61P 21/00

(52) U.S. Cl. CPC A61K 9/0024; A61K 31/506; A61K 47/34; A61K 47/42; A61P 21/00

(57) ABSTRACT

Methods of repairing composite tissue injuries susceptible to adipocyte infiltration, by applying to the injury an electrospun polymer fiber scaffold comprising biocompatible and bioresorbable polymer fibers that are blended with an amount of a GSK-3 inhibitor that is effective to inhibit the infiltration of adipocytes into the scaffold and tissue. Polymer scaffolds blended with an amount of a GSK-3 inhibitor that is effective to inhibit the infiltration of adipocytes into the scaffold and tissue are also disclosed as are kits containing the scaffolds and stem cells for repairing tissue at the injury site.

### CROSS-REFERENCE TO RELATED APPLICATIONS

This application is entitled to priority pursuant to 35 U.S.C. § 119(e) to U.S. Provisional Application No. 63/574,120, filed on Apr. 3, 2024. The content of the application is incorporated herein by reference in its entirety.

### FIELD OF THE INVENTION

This invention relates to polymeric scaffolds that comprise a GSK-3 (glycogen synthase kinase-3) inhibitor and methods of use thereof to treat or repair composite tissue injuries susceptible to adipocyte infiltration such as myotendinous junction (MTJ) injuries. This invention also relates to a method of preventing fatty infiltration of the myotendinous junction.

### BACKGROUND OF THE INVENTION

Muscle injuries are the most common mechanism of injury in athletes accounting for approximately 10% to 55% of all injuries. Traumatic lesions are classified as either direct or indirect injuries. Indirect trauma is caused by sudden forced lengthening over the viscoelastic limits of muscles during contraction while direct trauma is the result of an external force applied to the muscle. Strain injuries are the most encountered indirect trauma in professional sports and are defined as muscular trauma affecting the tissue structure with various degrees of tear. The main site of strain injury is the musculotendinous junction. Muscle groups that are exposed to rapid stretching such as hamstrings, calves, and rectus femoris are most susceptible to strain injuries. Injuries to these muscle groups occur during activities where the muscle is passively over-stretched or during fast eccentric contractions such as kicking and sprinting activities. However, strain injuries located at the musculotendinous junction can also occur in the upper body such as the rotator cuff(RC). Athletes competing in overhead sports such as baseball pitchers are prone to RC tears. Fatty accumulation within and around the muscle is commonly seen in severe rotator cuff tears (RCTs), which impairs muscle strength. RCTs near the myotendinous junction (MTJ), present a greater risk of reinjury and longer rehabilitation rates. Moreover, the rate of recurrent tears is correlated with the severity of fatty degeneration.

Magnetic resonance imaging (MRI) is used to examine the location of muscle injuries and identify the severity of the injury. The grades of severity are as follows: (1) no disruption of muscle fibers, (2) partial tear of the MTJ with hematoma, and (3) complete disruption of the MTJ causing loss of muscle function with extensive edema and hemorrhage. Significant injuries that require medical treatment are concerned with a specialized region that is responsible for the transmission of contractile force from muscle to skeleton, the MTJ. At the MTJ, the force generated by the muscle is transmitted from muscle filaments to the collagen fibers of the tendon tissue. The highly specialized morphology of MTJ allows it to withstand great mechanical stress. The 3-D reconstructive electron microscopy of the human MTJ has demonstrated that the collagen fibrils insert into the indentation of the muscle with extensive folding creating finger-like processes. The finger-like processes maximize the surface area of the interface, thereby reducing stress and increasing the strength at the site of force transmission. However, the transmission of extensive forces, especially during eccentric loading, can cause severe injuries such as RC tears, Achilles tendon ruptures, and hamstring damage of the MTJ during repetitive and heavy muscle activity. The structural damage to the muscle fibers may be caused by a single contraction or by the cumulative effect of several contractions. Muscle strain injuries are characterized by a sudden onset of pain which depending on severity may immediately prevent the athlete from continuing the sport activity. The potential decrease in the risk of strain injuries has been associated with the spatial changes that occur at the MTJ with exercise. It has been reported that training results in an increase in interdigitations at the MTJ which increases the contact area between muscle and tendon. On the other hand, unloaded muscle exhibits a decrease in interdigitations which increases the risk of injury. Therefore, following periods with lower loading the risk of strain injury is increased when high loading is resumed. Additionally, age predisposes to muscle strain injuries due to the shortening of interdigitations at the muscle-tendon interface thus resulting in muscle atrophy. Injuries to the MTJ have a longer recovery period. Recovery time needed for the injured muscle to regain functionality keeps athletes from training and competing.

Strain injuries located at the MTJ are subject to surgical suturing methods that present a greater risk of re-injury and longer rehabilitation times. Reported incidents of MTJ tears are mainly concerned with the surgical repairing techniques used to treat ruptures of the RC. Medial tears of the RC that occur at the MTJ are classified into 3 patterns: (1) Type A tears exhibit healthy tendon and muscle, (2) Type B exhibits healthy muscle laterally, but the remaining medial tendon is short and retracted, and (3) Type C tears occur when the tears become chronic. Patients with Type C tears need reconstructive procedures to restore the functionality of the native tissue. Type C tears are severe injuries and may have tendon remaining at the footprint but the muscle is retracted, is of insufficient length, and has fatty infiltration. Fatty accumulation within and around the muscles is encountered after RC tears due to loss of tension at the MTJ and has commonly been reported in MTJ tears of the supraspinatus muscle. The number of fibro-adipogenic progenitors (FAPs) in the RC is higher than that of the quadricep muscles, which could explain the degree of fat accumulation reported in supraspinatus muscle tears as opposed to muscles in the lower limb. Fatty infiltration of the muscle-tendon unit prevents the formation of healthy tissue. In the event of injury, the proliferative adipocytes accumulate at the site of injury hindering the proliferation of muscle and tendon cells. This disrupts the native structure of the MTJ compromising its functionality. Muscle strength is impaired by the formation of adipose tissue. Surgical repair of MTJ is challenging due to high rates of clinical and structural failure. Fat accumulation at the MTJ extends recovery periods and increases the susceptibility to reinjuries. Approximately 4 out of 5 re-injuries occur at the same site as the original injury, indicating suboptimal healing of the MTJ injury.

In muscle injury, muscle stem cells, also known as satellite cells (MuSCs), are activated to become proliferative myoblasts. Myoblasts differentiate and fuse to form multinucleated myotubes/myofibers which reconstruct the fibrous network of muscle tissue. Muscle injury activates other cell types that guide MuSCs through myogenesis. Recent studies have identified FAPs as regulators of skeletal muscle regeneration. FAPs are muscle-specific mesenchymal stromal cells that can differentiate into fibroblasts and adipocytes. FAPs found in the interstitial space of resting or regenerating skeletal muscle are inactive. Activation of FAPs contributes to muscle homeostasis and regeneration by supporting the differentiation of MuSCs. Once an injury occurs, cytokines such as IL-4 and/or IL-15 stimulate quiescent FAPs to divide and migrate to the injury site. Upon activation, FAPs express secretion factors that signal MuSCs to differentiate. Although FAPs play a crucial role in muscle regeneration, they are recognized for their ability to infiltrate adipocytes into skeletal muscle in acute injury. Fatty infiltration of the muscle is common in diseases including obesity, type 2 diabetes, and muscle deterioration during aging. Researchers have been investigating the role of WNT/β-catenin-dependent signaling, referred to as the “canonical” pathway, in FAP differentiation into adipocytes. The canonical pathway has been shown to inhibit adipogenesis and stimulate myogenesis and muscle glucose uptake in multiple studies, particularly concerned with diabetes. Wingless-related integration site (WNT) proteins are cysteine-rich glycoproteins that regulate stemness, self-renewal, migration, and differentiation of MuSCs. In the absence of a WNT molecule, a degradation complex consisting of glycogen synthase kinase 3 β (GSK-3β), adenomatous polyposis coli (APC), and Axin decreases β-catenin levels due to GSK-3-dependent phosphorylation. WNT binding to a receptor complex consisting of a Frizzled receptor protein and a low-density lipoprotein-related peptide (Lrp5/6), disintegrates the degradation complex. Upon binding to the Lrp5/6, the Frizzled receptor complex becomes phosphorylated creating a binding site for Axin. Recruitment of Axin inhibits GSK-3-mediated phosphorylation of β-catenin, allowing β-catenin levels to increase. This results in the accumulation of cytosolic β-catenin and its translocation to the nucleus in which it binds to the lymphoid enhancer-binding factor/T-cell-specific transcription factor (LEF/TCF) to activate WNT target genes. The upregulation of β-catenin suppresses proliferator-activated receptor gamma (PPARγ) expression. PPARγ is a regulator of adipogenesis and is upregulated three days after injury which corresponds to the time where FAPs expand to support myogenesis. These findings suggest controlling β-catenin levels is crucial in balancing the beneficial and detrimental effects of FAPs in muscle regeneration.

Tissue engineering approaches are frequently used to create single tissue types. A commonly used strategy is to combine an appropriate cell type with a suitable biodegradable scaffold to generate functional tissues in vivo. Material type and scaffold composition play an important role in determining cell behavior in response to the scaffold. Electrospinning is a popular scaffold fabrication method that allows control over scaffold structure, mechanical properties, and composition. The fibrous structure of electrospun scaffolds resembles the extracellular matrix (ECM), thereby demonstrating the morphology and mechanical properties of biological tissues. Significant progress has been made in promoting tissue growth through synthetic polymers for tissues including bone, tendon, and muscle. However, only a few studies have focused on the fabrication of scaffolds for composite tissue injuries such as MTJ tears. In addition, current research on muscle-tendon regeneration overlooks the effects of potential fat buildup. FAPs adipogenic differentiation can interfere with the proliferation rates of myoblasts and tenocytes at the site of injury causing scaffold malfunction.

### SUMMARY OF THE INVENTION

In one aspect, provided are polymeric scaffolds that comprise a GSK-3 (glycogen synthase kinase-3) inhibitor and methods of use thereof.

In one aspect, provided is an electrospun polymer fiber scaffold comprising a biocompatible and bioresorbable polymer fibers that are blended with an amount of a GSK-3 inhibitor. In some embodiments, the polymer is selected from the group consisting of poly(lactic-co-glycolic acid) (PLGA), poly-(glycolic acid) (PGA), polycaprolactone (PCL), polyesteramide (PEA), polyphosphazene, and poly(L-lactic acid) (PLA). In one embodiment, the polymer is soaked in or coated with gelatin. In one embodiment, the gelatin comprises cross-linked gelatin methacrylate. In one embodiment, the scaffold comprises collagen fibers. In some embodiments, the PCL to collagen ratio ranges from about 1:1 to about 4:1. In some embodiments, the amount of the GSK-3 inhibitor comprises 0.5 μM to 2.0 μM. In some embodiments, the scaffold comprises pores of about 200 μm to about 500 μm in diameter. In one embodiment, the GSK-3 inhibitor is CHIR99021.

In some embodiments, provided is a kit comprising the electrospun polymer fiber scaffold described herein. In some embodiments, the kit further comprises muscle stem cells (MuSCs).

In one aspect, provided is a method of repairing a composite tissue injury susceptible to adipocyte infiltration, comprising applying to the injury an electrospun polymer fiber scaffold comprising biocompatible and bioresorbable polymer fibers that are blended with an amount of a GSK-3 inhibitor that is effective to inhibit the infiltration of adipocytes into the scaffold and tissue. In one embodiment, the polymer is soaked in or coated with gelatin. In one embodiment, the gelatin comprises cross-linked gelatin methacrylate. In one embodiment, the composite tissue injury susceptible to adipocyte infiltration is a myotendinous junction (MTJ) injury. In one embodiment, MTJ injury is a rotator cuff medial tear. In some embodiments, the tear is a type A tear, a type B tear, or a type C tear.

In one aspect, provided is a method of preventing infiltration of adipocytes into a scaffold and tissue following a composite tissue injury, comprising administering to a subject in need thereof an electrospun polymer fiber scaffold comprising biocompatible and bioresorbable polymer fibers that are blended with a GSK-3 inhibitor that is effective to inhibit the infiltration of adipocytes into the scaffold and tissue. In some embodiments, the polymer is selected from the group consisting of poly(lactic-co-glycolic acid) (PLGA), poly-(glycolic acid) (PGA), polycaprolactone (PCL), polyesteramide (PEA), polyphosphazene, and poly(L-lactic acid) (PLA). In one embodiment, the composite tissue injury is a MTJ injury.

### BRIEF DESCRIPTION OF THE FIGURES

FIGS. 1A and 1B depict graphs of Oil Red O. quantification in adipose-derived stem cells (ADSCs) and bone marrow-derived human mesenchymal stem cells (hMSCs), demonstrating that CHIR990021 decreases lipid droplet formation in ADSCs and hMSCs. FIG. 1A is a graph of the results of ADSCs after adipogenic induction. ADSCs showed significant difference in lipid formation in groups treated with CHIR99021 at 21 days (One-Way ANOVA, P=0.00003). GM=growth medium; ADM (adipogenic differentiation medium)=GM supplemented with 0.1 μM dexamethasone, 1 g/mL insulin, 0.2 mM indomethacin, 0.45 mM IBMX, 1 μM rosiglitazone; CHIR99021 (“CHIR”) concentrations in ADM were adjusted to 0.5 μM, 1.0 μM, and 1.5 μM. FIG. 1B is a graph of the results of hMSCs treated at days 14 and 18. hMSCs showed significant difference in lipid formation in groups treated with CHIR99021 at 18 day (One-Way ANOVA, P=0.0000018).

FIGS. 2A and 2B demonstrate that CHIR99021 does not negatively alter cellular metabolic activity or viability of hMSCs. FIG. 2A is a graph of PrestoBlue™ analysis of hMSCs at days 7, 14, and 18 treated with GM, ADM, or the indicated concentrations of CHIR99021 (“CHIR”). CHIR concentrations in ADM were adjusted to 0.5 μM, 1.0 μM, and 1.5 μM. FIG. 2B is a graph of the percent of live hMSCs from Live/Dead quantification at days 7, 14, and 18 that were treated as indicated. Live/Dead quantification was performed on separate plates for each time point, thus limiting the ability to track trends in live cell counts over time.

FIGS. 3A-3C illustrate that CHIR99021 does not negatively alter cellular metabolic activity or viability of ADSCs. FIG. 3A is a graph of PrestoBlue™ analysis of ADSCs at days 3, 7, 14, and 21 treated with GM, ADM, or the indicated concentrations of CHIR99021 (“CHIR”). CHIR concentrations in ADM were adjusted to 0.5 μM, 1.0 μM, and 1.5 μM. FIG. 3B is a graph of the percent of live ADSCs from Live/Dead quantification at days 7, 14, and 21 treated with GM, ADM, or the indicated concentrations of CHIR. FIG. 3C is a graph of the percent of dead ADSCs at day 21 treated with GM, ADM, or the indicated concentrations of CHIR. Cells were stained with bisbenzimide (Hoechst 33342) for total nucleus counts revealing no significant difference in % dead cells between ADSCs in GM and in 1.5 μM CHIR at day 21 (t-test, P(T<=t) two-tail=0.46).

FIGS. 4A and 4B illustrate the CHIR99021 release profile as depicted by high-performance liquid chromatography (HPLC). CHIR99021 release from PLGA nanofibers follows a first-order release. FIG. 4A is a graph of the cumulative percent of drug release. FIG. 4B is a graph of the percent of remaining drug.

FIGS. 5A and 5B are graphs of PrestoBlue™ fluorescence in human skeletal muscle cells (hSkMCs) at day 4 or day 7 cultured in the indicated conditions. PrestoBlue™ fluorescence was measured at an excitation wavelength 560 nm and emission wavelength of 590 nm. FIG. 5A is a graph of PrestoBlue™ fluorescence in hSkMCs treated with PLGA/GelMA-CHIR in ADM, PLGA-CHIR in ADM, PLGA/GelMA, PLGA alone in ADM, ADM without a scaffold, and growth medium without a scaffold (GM). FIG. 5B is a graph of PrestoBlue™ fluorescence in hSkMCs treated with PLGA/GelMA-CHIR in ADM, PLGA-CHIR in ADM, PLGA/GelMA, myogenic differentiation media (MYODIFF) without a scaffold, and GM without a scaffold. ADM promotes the formation of lipid droplets while MYODIFF promotes the formation of myotubes.

### DETAILED DESCRIPTION OF THE INVENTION

### 1. Overview

The present disclosure provides methods for treating a composite tissue injury susceptible to adipocyte infiltration such as a myotendinous junction (MTJ) injury. In one embodiment, the composite tissue injury is a muscle injury. In one aspect, provided are methods for treating a composite tissue injury comprising administering to a subject in need thereof a polymeric scaffold, wherein the scaffold comprises a GSK-3 (glycogen synthase kinase-3) inhibitor. In another aspect, provided is a biocompatible and bioresorbable polymer mesh scaffold loaded with a GSK-3 inhibitor. In another aspect, provided is a method of preventing infiltration of adipocytes into a muscle, comprising administering to a subject in need thereof a polymeric scaffold, wherein the scaffold comprises a GSK-3 inhibitor. In one embodiment, a polymeric scaffold described herein inhibits GSK-3β, which in turn suppresses PPARγ, a regulator of adipogenesis, by increasing β-catenin levels.

### 2. GSK-3 and GSK-3 Inhibitors

GSK-3 is a serine/threonine kinase that is involved in a variety of cellular processes (e.g., coordinating catabolic and anabolic pathways). GSK-3 exists as two isozymes: GSK-3a and GSK-3β. In the absence of a WNT molecule, β-catenin is phosphorylated by GSK-3β in the destruction complex, which leads to the degradation of β-catenin levels. In contrast, during WNT stimulation, the recruitment of Axin to the membrane prevents β-catenin phosphorylation (Law, S. M. et al. Premise and peril of Wnt signaling activation through GSK-3beta inhibition. iScience, 2022. 25(4): p. 104159). As a result, β-catenin translocates to the nucleus to initiate the transcription of WNT target genes (Rudnicki, M. A. et al., Wnt signaling in bone and muscle. Bone, 2015. 80: p. 60-66). Inhibition of GSK-3β can emulate the removal of Axin with WNT signaling leading to the accumulation of β-catenin (Law, S. M. et al., Premise and peril of Wnt signaling activation through GSK-3beta inhibition. iScience, 2022. 25(4): p. 104159). GSK-3 inhibitors increase β-catenin levels, which in turn suppresses PPARγ expression. Fatty infiltration of the muscle is commonly observed in diabetes. In diabetic patients, administration of GSK-3 inhibitors has been reported to reduce glycerol-induced intramuscular fat formation by suppressing PPARγ expression in FAPs (Giuliani, G. et al., Signaling pathways regulating the fate of fibro/adipogenic progenitors (FAPs) in skeletal muscle regeneration and disease. The FEBS Journal, 2022. 289(21): p. 6484-6517).

Many GSK-3β inhibitors with therapeutic potentials can stimulate WNT signaling. For instance, CHIR99021 is widely used as a GSK-3β inhibitor to activate WNT signaling and inhibit adipogenesis. It was reported CHIR99021 induced PPARγ expression and blocked the differentiation of 3T3-L1 preadipocytes as assessed by Oil Red O staining (Bennett, C. N., et al., Regulation of Wnt signaling during adipogenesis. J Biol Chem, 2002. 277(34): p. 30998-1004).

CHIR99021 has the formula:

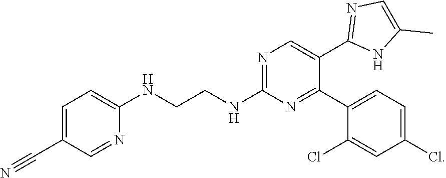 [SMILES: Cc1cnc(-c2cnc(NCCNc3ccc(C#N)cn3)nc2-c2ccc(Cl)cc2Cl)[nH]1]

Essentially any GSK-3 inhibitor can be used that effectively inhibits GSK-3a or GSK-3β kinase activity. In one embodiment, the GSK-3 inhibitor used in a polymeric scaffold described herein is a GSK-3a or GSK-3β inhibitor. Examples of GSK-3 inhibitors that can be used in the scaffold described herein, include, but are not limited to: 4-1, 4-2, 4-3, 4-4, 4-5, 5-imino-1,2,4-thiadiazole, AF3581, Alsterpaullone, AR-A014418, Azakenpaullone, AZD1080, AZD2858, BIO, BIP-135, BRD0705, BRD3731, Cazpaullone, CHIR98014, CHIR98023, CHIR99021, GSK-3β Inhibitor VI, indirubin-3′-oxime, IMID1, IMID2, JGK-263, Kenpaullone, L803mt, L807mts, MMBO, PF-04802367 (PF-367), ruboxistaurin, SAR502250, SB-216763, SB-415286, SC100, TCS2002, TDZD-8, Tideglusib (NP031112, NP-12), TWS119, VP0.7, VP 1.14, VP 1.16, VP2.51, VP2.54, and VP3.35. In one embodiment, the GSK-3 inhibitor is CHIR99021.

In some embodiments, the scaffold described herein comprises an amount of GSK-3 inhibitor that ranges from 0.5 μM to 5.0 μM such as 0.5 μM, 1.0 μM, 1.5 μM, 2.0 μM, 2.5 μM, 3.0 μM, 3.5 μM, 4.0 μM, 4.5 μM, 5.0 μM or any amount therebetween. In some embodiments, the amount of the GSK-3 inhibitor ranges from 0.5 uM to 2.0 uM such as 0.5 μM, 1.0 μM, 1.5 μM, 2.0 μM or any amount therebetween.

### 3. Scaffolds

Provided herein are electrospun or fibrous polymeric scaffolds loaded with a GSK-3 inhibitor described herein. The GSK-3 inhibitor is combined with the polymer in an electrospinning solvent prior to electrospinning. In some embodiments, the electrospinning solvent contains 1.0 μM to 3.0 μM of a GSK-3 inhibitor such as 1.0 μM, 1.5 μM, 2.0 μM, 2.5 μM, 3.0 μM, or any concentration therebetween. In one embodiment, the electrospinning solvent contains 1.5 μM of a GSK-3 inhibitor. In one embodiment, the electrospinning solvent contains 1.5 μM of CHIR99021. In one embodiment, the electrospinning solvent contains 3.0 μM of CHIR99021. In one embodiment, the concentration of CHIR99021 ranges from 1.4×10⁻³ to 2.4×10⁻³ grams/mL such as 1.4×10⁻³ grams/mL, 1.5×10⁻³ grams/mL, 1.6×10⁻³ grams/mL, 17×10⁻³ grams/mL, 1.8×10⁻³ grams/mL, 1.9×10⁻³ grams/mL, 2.0×10⁻³ grams/mL, 2.1×10⁻³ grams/mL, 2.2×10⁻³ grams/mL, 2.3×10⁻³ grams/mL, 2.4×10⁻³ grams/mL or any concentration therebetween.

Synthetic biomaterials have good reproducibility and tunable chemical and physical properties, but require further modifications to their surface and structure to promote their biofunctionality. Natural materials such as collagen exhibit intrinsic similarity to ECM, but have lower mechanical strengths and faster degradation rates that limit in vitro culture, handleability during implantation, and resistance to in vivo forces. For this reason, synthetic and natural biomaterials are combined to enhance scaffolds' biocompatibility, mechanical, and structural properties. In one embodiment, PCL is a slow-degrading biocompatible polymer that is used in instances where long-term mechanical or structural support is desired. Further, crosslinking agents can be used to increase the mechanical strength of native materials such as collagen. Polymer/collagen scaffolds crosslinked by a carbodiimide-mediated coupling reaction, for example, using EDC/NHS (1-ethyl-3(3-dimethylaminopropyl-carbodiimide hydrochloride/N-hydroxysuccinimide) can be fabricated.

### Polymers

Essentially any biocompatible and bioresorbable polymer suitable for implantation in a subject and capable of being electrospun is suitable for use with the present invention. Electrospinnable polymers include those that are soluble in at least one organic solvent or water and have sufficiently high molecular weight to be above the “chain entanglement point,” which is defined as the minimum molecular weight needed for the polymer to form a self-supporting film by solvent casting. One of skill in the art is capable of determining the chain entanglement point of a polymer.

Examples of polymers used to generate the polymeric scaffolds described herein include, but are not limited to poly(lactic-co-glycolic acid) (PLGA), poly-(glycolic acid) (PGA), polycaprolactone (PCL), polyesteramide (PEA), polyphosphazene, and poly(L-lactic acid) (PLA) (Alaswad et al., Polymers (Basel). 2022. 14(22): 4924 and BaoLin, Guo et al. Sci China Chem. 2014. 57(4): 490-500). Additional exemplary polymers include, but are not limited to polysaccharides, poly(alkylene oxides), polyarylates, for example those disclosed in U.S. Pat. No. 5,216,115, block co-polymers of poly(alkylene oxides) with polycarbonates and polyarylates, for example those disclosed in U.S. Pat. No. 5,658,995, polycarbonates and polyarylates, for example those disclosed in U.S. Pat. No. 5,670,602, free acid polycarbonates and polyarylates, for example those disclosed in U.S. Pat. No. 6,120,491, polyamide carbonates and polyester amides of hydroxy acids, for example those disclosed in U.S. Pat. No. 6,284,862, polymers of L-tyrosine derived diphenol compounds, including polythiocarbonates and polyethers, for example those disclosed in U.S. Pat. No. RE37,795, strictly alternating poly(alkylene oxide) ethers, for example those disclosed in U.S. Pat. No. 6,602,497, polymers listed on the United States FDA “EAFUS” list, including polyacrylamide, polyacrylamide resin, modified poly(acrylic acid-co-hypophosphite), sodium salt polyacrylic acid, sodium salt poly(alkyl(C16-22) acrylate), polydextrose, poly(divinylbenzene-co-ethylstyrene), poly(divinylbenzene-co-trimethyl(vinylbenzyl)ammonium chloride), polyethylene (m.w. 2,00-21,000), polyethylene glycol, polyethylene glycol (400) dioleate, polyethylene (oxidized), polyethyleneimine reaction product with 1,2-dichloroethane, polyglycerol esters of fatty acids, polyglyceryl phthalate ester of coconut oil fatty acids, polyisobutylene (min. m.w. 37,000), polylimonene, polymaleic acid, polymaleic acid, sodium salt, poly(maleic anhydride), sodium salt, polyoxyethylene dioleate, polyoxyethylene (600) dioleate, polyoxyethylene (600) mono-ricinoleate, polyoxyethylene 40 monostearate, polypropylene glycol (m.w. 1,200-3,000), polysorbate 20, polysorbate 60, polysorbate 65, polysorbate 80, polystyrene, cross-linked, chloromethylated, then aminated with trimethylamine, dimethylamine, diethylenetriamine, or triethanolamine, polyvinyl acetate, polyvinyl alcohol, polyvinyl pyrrolidone, and polyvinylpyrrolidone, and polymers listed in U.S. Pat. No. 7,112,417, the disclosures of all of which are incorporated herein by reference in their entirety.

In one embodiment, the polymer is PCL. PCL is a slow-degrading biocompatible polymer that is used in instances where long-term mechanical or structural support is desired.

In one embodiment the polymeric scaffolds described herein further comprise collagen fibers. For example, the incorporation of type I collagen fibers in polymer scaffolds, such as PCL scaffolds, can improve cell adhesion and growth in skeletal muscle regeneration (See, Politi, S. et al. Smart ECM-Based Electrospun Biomaterials for Skeletal Muscle Regeneration. Nanomaterials (Basel), 2020. 10(9) and Choi, J. S. et al. The influence of electrospun aligned poly(c caprolactone)/collagen nanofiber meshes on the formation of self-aligned skeletal muscle myotubes. Biomaterials, 2008. 29(19): p. 2899-2906). In one embodiment, PCL/collagen scaffolds are crosslinked with EDC/NHS. PCL and collagen type I can be dissolved in an electrospinning solvent such as 1,1,1,3,3,3-Hexafluoro-2-propanol (HFP), each at concentrations of 15% (w/v).

In collagen-containing scaffold embodiments, the collagen is included in the electrospinning solvent containing the polymer and the GSK-3 inhibitor. Further, crosslinking agents can be used to increase the mechanical strength of native materials such as collagen. Polymers with reactive amine or carboxylic acid groups can be cross-linked with collagen via carbodiimide mediated coupling reactions in the electrospinning solvent in the presence of NHS to limit side reactions and increase yield.

In one embodiment, the polymer is soaked in or coated with gelatin. Gelatin increases biocompatibility and a GSK-3 inhibitor described herein can be encapsulated inside of a gelatin hydrogel. In one embodiment, the gelating hydrogel is formed using gelatin methacrylate that is crosslinked by conventional means. Crosslinking the GSK-3 inhibitor within the hydrogel using gelatin methacrylate creates a prolonged release as the drug release occurs due to normal diffusion or by cells degrading the gelatin methacrylate and releasing the drug.

As used herein, the term “gelatin” refers to gelatin type A. In one embodiment, the gelatin is type A porcine skin gelatin.

In one embodiment, a scaffold described herein comprises 100% PCL scaffolds soaked in gelatin. In one embodiment, a scaffold described herein comprises a PCL/collagen weight ratio of 1:1. In one embodiment, scaffolds described herein comprise a PCL/collagen weight ratio ranging from about 1:1 to about 4:1.

In one embodiment, the polymeric scaffolds described herein are biodegradable.

### Electrospinning

The polymeric scaffolds described herein are generated via electrospinning as known in the art. Electrospinning is a popular tissue engineering approach to creating single tissue types. It can be a solvent or melt process, with the solvent process being attractive for biocompatible polymers that are not melt-processable. Any solvent suitable for use with electrospinning can be selected, such as HFP. In an electrospinning setup, nanofibers are formed through the stretching of a viscoelastic solution in the presence of an external electric field. The process requires a syringe charged with a polymeric solution provided with a metallic needle, a syringe pump, a high-voltage power supply, and a collector. The syringe's metallic needle is charged by connecting to the high-voltage power supply. As a result of surface tension, spherical droplets are formed upon extrusion of the polymeric solution from syringes. The droplets deform into a conical shape, a Taylor cone, when the repulsion exceeds the surface tension, and a charged jet is drawn to the collector. The charge on the jet causes the polymer chains inside the solution to stretch leading to a whipping motion, referred to as bending instability. As the jet is stretched, the polymer solution starts solidifying leading to the formation of randomly oriented nanofibers on the collector. Fiber alignment is crucial in scaffolds that are intended to promote muscle and tendon regeneration. Several electrospinning parameters including the rate of the flow pump, applied voltage, distance between the needle and the collector, and shape and movement of the collector influence fiber morphology, all of which can be readily ascertained by one of ordinary skill in the art.

In one embodiment, an electrospinning solution comprises polymer, collagen, and a GSK-3 inhibitor as described herein. In one embodiment, polymer/collagen scaffolds are crosslinked with carbodiimide mediated coupling. In one embodiment, the polymer is soaked in gelatin after electrospinning. In one embodiment, PCL and collagen type I are dissolved in electrospinning solvent, each at concentrations of 15% (w/v).

In one embodiment, the electrospinning further comprises co-spinning a sacrificial polymer with polymer/collagen scaffolds (i.e., structural component) and removal of the sacrificial polymer (i.e., sacrificial component). Sacrificial fibers are introduced to create a range of pore sizes intended to promote cell growth. In this case, two flow pumps are connected to different positive voltage supplies to extrude the polymer/collagen and sacrificial solutions from different syringes. Like charges cause repulsions because the needles are connected to positive voltages, which in turn prevents the formation of a scaffold composed of both polymers (i.e., sacrificial and structural polymers). Flow pumps are connected to the different positive voltage supplies on opposite sides of a cylindrical drum.

In one embodiment, the sacrificial polymer is polyethylene oxide (PEO). PEO is a water-soluble polymer that is nontoxic to cells. For example, PEO is dissolved in 100% ethanol at room temperature on a vortexer to yield a 10% (w/v) solution. In one embodiment, the sacrificial polymer is polyglycolic acid. Incorporating sacrificial fibers in an electrospun mesh and their subsequent removal to increase porosity of the mesh is essentially conventional and well-understood by those of ordinary skill in the art.

The fibrous structure of electrospun scaffolds demonstrates the morphology and mechanical properties of biological tissues. The orientation of fibers within electrospun scaffolds influences cell behavior. Fabrication of aligned nanofibers through electrospinning show promise for fiber-reinforced tissues. For example, fiber alignment in native muscle allows cells to form aligned myotubes during skeletal muscle regeneration. Similarly, fiber alignment in tendons optimizes their load-bearing ability. Altering electrospinning parameters enables the creation of scaffolds with aligned nanofibers to support the growth of muscle and tendon cells.

Although favorable in mechanical properties, electrospun scaffolds are limited in their ability to promote cell infiltration due to the presence of highly dense nanofibers, particularly true for aligned nanofibers. Increasing porosity inside the scaffold can increase cell infiltration. This porosity allows for nutrients and oxygens to enter the scaffold and for waste products to exit to support tissue growth. In one embodiment, the pore size ranges from 200 μm to 500 μm such as 200 μm, 300 μm, 400 μm, 500 μm, or any size therebetween.

### Additional Therapeutic Agents

In some embodiments, the polymeric scaffolds comprise an additional therapeutic agent. In one embodiment, the scaffolds described herein comprise an antibiotic and/or anti-inflammatory agents. Examples of therapeutic agents include, but are not limited to growth factors (such as fibroblast growth factor (FGF) or vascular endothelial growth factor (VEGF)), non-steroidal anti-inflammatory drugs (NSAIDs), and antibiotics such as penicillin, ampicillin, and the like. Examples of NSAIDs include, but are not limited to aspirin, ibuprofen, and naproxen.

### 4. Kit

In some embodiments, provided is a kit that comprises a polymeric scaffold described herein. In some embodiments, the kit further comprises MuSCs. The kit can further include instructions for use. In some embodiments, the instructions are for using a polymeric scaffold described herein to repair composite tissue injuries.

In one embodiment, provided is a kit comprising an electrospun polymer fiber scaffold according to the present invention, wherein the polymer is a biocompatible and bioresorbable polymer that is blended with a GSK-3 inhibitor as described herein. In one embodiment, the kit comprises PLA and the GSK-3 inhibitor is CHIR99021. In one embodiment, the kit comprises CHIR99021 and the polymer is PCL that is soaked in gelatin. In some embodiments, the kit comprises 0.5.0 μM to 5.0 μM of the GSK-3 inhibitor such as 0.5.0 μM, 1.0 μM, 1.5 μM, 2.0 μM, 2.5 μM, 3.0 μM, 3.5 μM, 4.0 μM, 4.5 μM, 5.0 μM or any amount therebetween. In some embodiments, the kit comprises 0.5 μM to 2.0 uM of the GSK-3 inhibitor such as 0.5 μM, 1.0 μM, 1.5 μM, 2.0 μM or any amount therebetween.

### 5. Methods of Treatment

In some embodiments, the polymeric scaffolds described herein are used to treat or repair a composite tissue injury susceptible to adipocyte infiltration in a subject in need thereof. As used herein, the term “composite tissue injury” refers to an injury to any combination of bone, nerve, tendon, and soft tissue. Examples of composite tissue injuries susceptible to adipocyte infiltration include, but are not limited to a myotendinous junction (MTJ) injury. In one embodiment, the composite tissue injury is a MTJ injury. In one embodiment, the MTJ injury is a rotator cuff medial tear. In one embodiment, the tear is a type A tear, a type B tear, or a type C tear.

As used herein, the term “subject” refers to an animal, preferably a mammal such as a human.

As used herein the terms “treating” or “treatment” refers to administration of a polymeric scaffold described herein to a subject who has experienced a composite tissue injury susceptible to adipocyte infiltration.

As used herein the terms “repair” or “repairing” refers to administration of a polymeric scaffold described herein to a subject who has experienced a composite tissue injury susceptible to adipocyte infiltration to promote the promyogenic ability of FAPs (in other words, to promote the formation of muscle tissue).

In one embodiment, the polymeric scaffolds described herein are implanted into the injured muscle of a subject such as that following a composite tissue injury. In one embodiment, the damaged area in a subject is sutured together and the scaffold is wrapped around the outside of the damaged area and sutured to the tissue. In another embodiment, the scaffold is inserted inside of the damaged area and the damaged area sutured closed. In a further embodiment, another scaffold is wrapped around the outside of the damaged area and sutured to the tissue.

In one embodiment, the scaffold is loaded with muscle stem cells alone or with fibroblasts.

In one embodiment, the polymeric scaffolds described herein are used to prevent infiltration of adipocytes into muscle in a subject.

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

As used herein, the singular forms “a,” “an,” and “the” include plural references unless the context clearly dictates otherwise.

As used herein, the terms “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like are used repeatedly. Such phrases do not necessarily refer to the same embodiment, but they may unless the context dictates otherwise.

As used herein, the terms “and/or” or “/” means any one of the items, any combination of the items, or all of the items with which this term is associated.

As used herein, the word “substantially” does not exclude “completely,” e.g., a composition that is “substantially free” from Y may be completely free from Y. Where necessary, the word “substantially” may be omitted from the definition of the disclosure.

As used herein, the term “each,” when used in reference to a collection of items, is intended to identify an individual item in the collection, but does not necessarily refer to every item in the collection. Exceptions can occur if explicit disclosure or context clearly dictates otherwise.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1. CHIR99021 Inhibits Lipid Formation in Adipogenic hMSC and ADSC Cultures

The effect of CHIR99021 on adipogenic differentiation of bone marrow-derived human mesenchymal stem cells (hMSCs) and adipose-derived stem cells (ADSCs) was examined as a treatment for fat accumulation after MTJ repair.

### Methods

hMSCs and ADSCs were seeded at a density of 18,000 cells/cm² in growth medium (GM) (MEM α, nucleosides, no ascorbic acid, 10% FBS, 1% P/S (penicillin-streptomycin)) and switched to adipogenic differentiation medium (ADM) (GM supplemented with 0.1 μM dexamethasone, 1 g/mL insulin, 0.2 mM indomethacin, 0.45 mM IBMX, 1 μM rosiglitazone) with or without CHIR99021. CHIR99021 concentrations in ADM were adjusted to 0.5 μM, 1.0 μM, and 1.5 μM. Rosiglitazone (AVANDIA®) was added to the adipogenic medium at a final concentration of 1 μM before use. A PrestoBlue™ assay and a LIVE/DEAD viability/cytotoxicity kit were utilized to assess cell viability. Live and dead cell counts were obtained using a Celigo Image Cytometer. Cells were fixed in 4% PFA (paraformaldehyde) and stained with Oil Red O to measure lipid formation as a sign of adipocyte differentiation. Lipid formation was quantified by assessing the absorbance of Oil Red O at 515 nm.

### Results

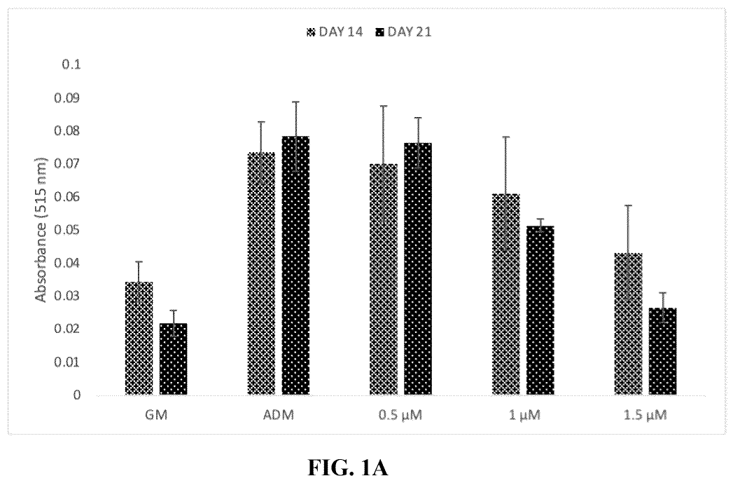

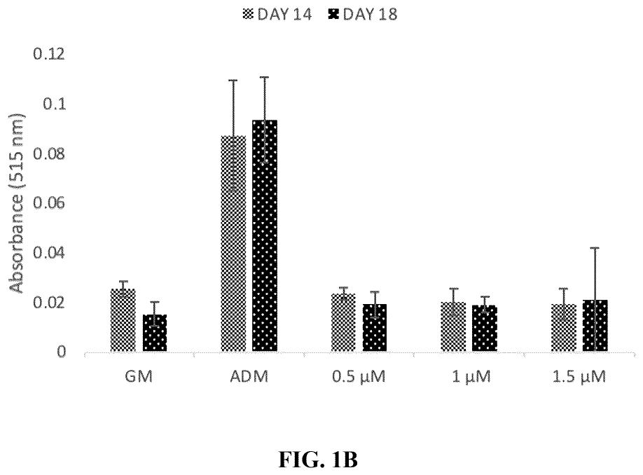

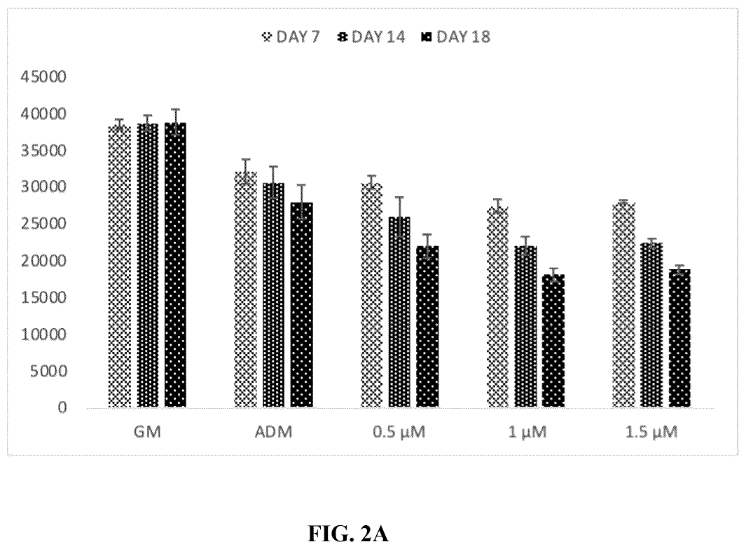

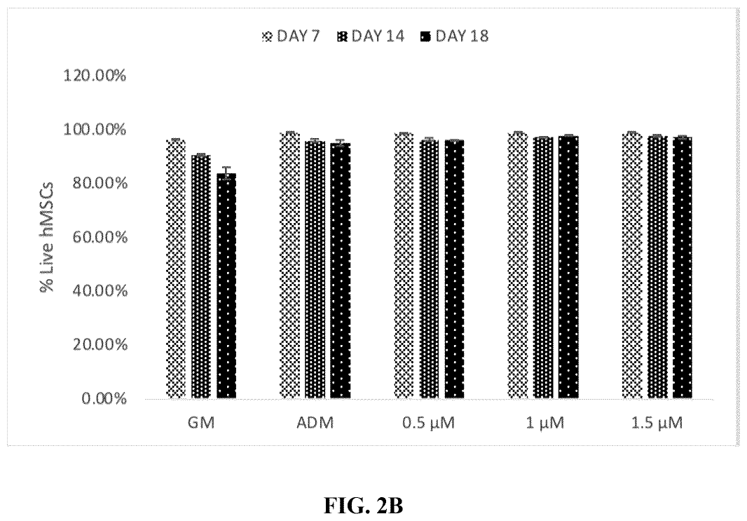

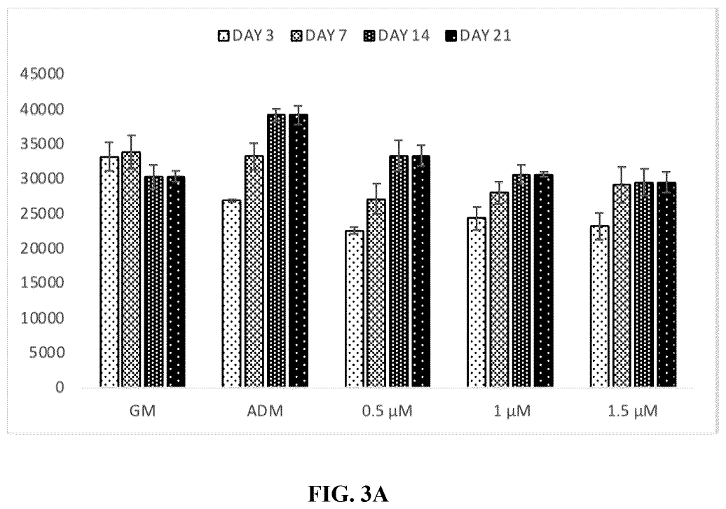

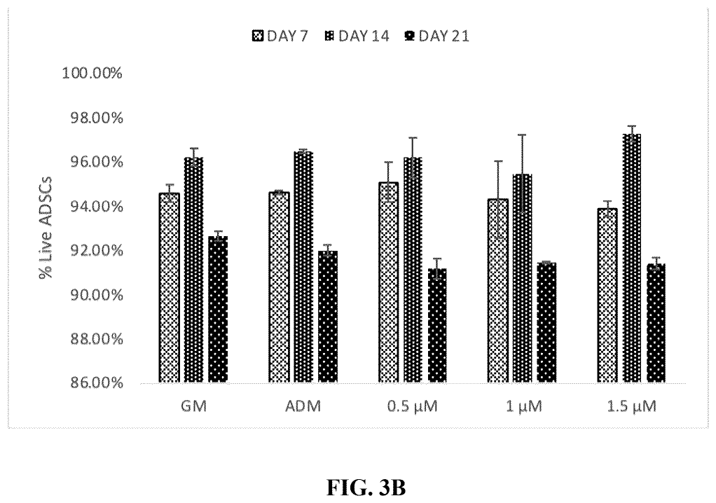

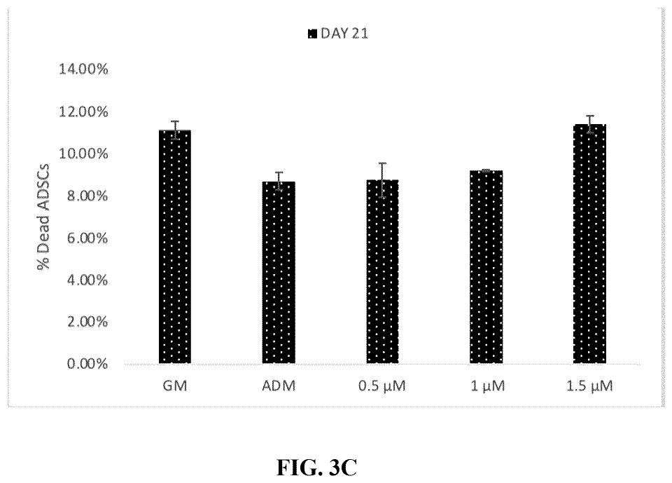

It was determined that CHIR99021 decreases lipid droplet formation in ADSCs and hMSCs (FIGS. 1A and 1B). The results obtained from PrestoBlue™ and Live/Dead assays, collected at day 7, day 14, and day 18 (FIGS. 2A and 2B) indicate that CHIR99021 does not negatively alter cellular metabolic activity or viability of hMSCs at 0.5 μM, 1.0 μM, and 1.5 μM. Moreover, the results obtained from PrestoBlue™ and Live/Dead assays, collected at day 7, day 14, and day 21 (FIGS. 3A and 3B) indicate that CHIR99021 does not negatively alter cellular metabolic activity or viability of ADSCs at 0.5 μM, 1.0 μM, and 1.5 μM. No significant difference was observed in the percent of dead cells between ADSCs in GM and in 1.5 μM CHIR at day 21 (FIG. 3C).

Administration of CHIR99021 in adipogenic cultures inhibited lipid formation (FIG. 1A). Cells cultured solely in ADM exhibit significantly higher lipid droplet formation compared to cells cultured with CHIR99021. Thus, CHIR99021 administration can inhibit lipid formation in adipogenic cultures of stem cells without inducing cell death at 0.5 μM, 1.0 μM, and 1.5 μM.

### Example 2. Preparation of PCL/Collagen Scaffolds with Polyethylene Oxide (PEO) Sacrificial Nanofibers

Scaffolds with sacrificial nanofibers are developed to promote muscle and tendon regeneration by achieving fiber alignment and creating pores that facilitate cell growth. Achieving fiber alignment is critical in mimicking the structural composition of native tendons and muscles. In native muscle, fiber alignment is essential for myoblasts to form aligned myotubes during myogenesis. Similarly, fiber alignment in tendons optimizes their load-bearing ability. Due to their role in movement, these tissues are prone to injuries. The formation of disorganized fibers during tendon regeneration interferes with tendons' mechanical ability and increases the risk of re-injuries. Electrospinning produces densely packed fibers, which negatively affects cell behavior in response to the scaffold. Generating fiber alignment and porosity while establishing PCL/collagen ratios that facilitate cell growth is achieved using co-electrospinning techniques to create scaffolds with sacrificial nanofibers.

### Methods:

### PCL Collagen Nanofibers:

Scaffolds with aligned PCL/collagen nanofibers were created to determine PCL/collagen ratios with the highest cell proliferation rates. Electrospinning was performed with PCL/collagen ratios of 100:0, 80:20, 60:40, 50:50, 40:60, 20:80, and 0:100. A single flow pump was used to extrude PCL/collagen blend from a 5 ml syringe with an 18-gauge stainless steel needle. A cylindrical drum with an adjustable rotation speed was used as the collector to create fiber alignment. The initial electrospinning parameters were set following a separate electrospinning experiment to create PCL scaffolds with PEO sacrificial fibers. The PEO was co-spun from a separate syringe. The distance between the needle and the collector was set to 15 cm, the potential is set to +13 kV and the flow rate is 1.0 ml/hr. These parameters were adjusted according to the characteristic of the polymer solution to produce fiber alignment without beads. Additionally, the collector was rotated at various speeds to determine its effect on fiber alignment. Fiber alignment and bead formation in PCL/collagen scaffolds was observed through scanning electron microscopy (SEM).

### Assessment of Collagen Degradation:

PCL/collagen scaffolds were crosslinked with EDC/NHS following the methods described by Chen et al. (Chen, D. et al., Electrospun polycaprolactone/collagen nanofibers cross-linked with 1-ethyl-3-(3-dimethylaminopropyl) carbodiimide/N-hydroxysuccinimide and genipin facilitate endothelial cell regeneration and may be a promising candidate for vascular scaffolds. Int J Nanomedicine, 2019. 14: p. 2127-2144). N-(3-dimethylaminopropyl)-N′-ethylcarbodiimide hydrochloride (EDC) is an easily removable crosslinking agent that achieves scaffold stability by covalently binding collagen molecules. EDC is often used in conjugation with N-hydroxysuccinimide (NHS) and has been shown to maintain electrospun collagen stability through in vitro and in vivo studies. A crosslinking solution was made of 2:1 EDC-to-NHS weight ratio using 95% ethanol as a solvent. Before crosslinking, scaffolds were incubated with 0.05 M MES buffer for 1 h and immersed in MES buffer containing EDC/NHS under gentle shaking for 24 h. Collagen degradation was assessed by measuring the initial weight of samples and then immersing them in PBS. Dry weights were measured at different time points to calculate the percentage of weight remaining.

### Assessment of Cell Viability and Proliferation:

The ability of different PCL/collagen ratios in supporting cell attachment and proliferation was assessed by conducting separate cell studies with MuSCs and fibroblasts. Cells were isolated from New Zealand white rabbits that were slaughtered. Biological activity of PCL/collagen scaffolds was assessed by testing the ability of cells to adhere and proliferate using an MTS assay (Promega) (Choi, J. S. et al. The influence of electrospun aligned poly(ε-caprolactone)/collagen nanofiber meshes on the formation of self-aligned skeletal muscle myotubes. Biomaterials, 2008. 29(19): p. 2899-2906) and Live/Dead assay (ab115347, Abcam) following the manufacturer's instructions after 1, 3, and 7 days of cell seeding.

To isolate MuSCs from the supraspinatus muscle, the methods described by Dohmen et al. and Ding et al. were adjusted. (Dohmen, R. G. J. et al. Muscle-derived fibro-adipogenic progenitor cells for production of cultured bovine adipose tissue. npj Science of Food, 2022. 6(1): p. 6 and Ding, S., et al., Maintaining bovine satellite cells stemness through p38 pathway. Scientific Reports, 2018. 8(1): p. 10808). The tissue was minced and dissociated with collagenase 2 in DMEM supplemented with 1% penicillin-streptomycin (PS). After digestion, cells were supplemented with DMEM containing 20% FBS. Muscle fragments were centrifuged, and the supernatant was collected as a cell suspension. Cell slurries were filtered through a 100 m cell strainer and incubated in ammonium-chloride-potassium (ACK) erythrocyte lysis buffer. The cell population was sorted into MuSCs by employing a fluorescence-activated cell sorting (FACS) strategy. Prior to FACS, cells were cultured for 72 h on bovine collagen type I coated flasks and sorted using antibodies, CD31 and CD45. CD31−/CD45− cells are sorted into a population of CD29+/CD56+ (MuSCs). Next, fibroblasts were isolated from the supraspinatus tendon following the methods described by Gögele et al. and Chard et al. (Gögele, C. et al. Cyclically stretched ACL fibroblasts emigrating from spheroids adapt their cytoskeleton and ligament-related expression profile. Cell Tissue Res, 2021. 384(3): p. 675-690 and Chard, M. D. et al. Isolation and growth characteristics of adult human tendon fibroblasts. Ann Rheum Dis, 1987. 46(5): p. 385-90). The tissue was minced and grown in medium (DMEM, 10% FBS, 1% PS) in a 37° C. incubator. Fibroblasts that adhered to the bottom of the culture dish were detached by trypsinization (0.05% trypsin-0.02% EDTA solution) (Id.).

Cells were seeded on scaffolds (10×10 mm²) at a density of 4×10⁴ cells/cm² and cultured in their respective growth medium in a 24-well plate: MuSCs in growth medium (Ham's F-10 Nutrient Mix, 20% heat-inactivated FBS, 5 ng/mL Recombinant human bFGF, and 1% PSA) and fibroblasts in DMEM with 10% FBS, 1% PS. For the MTS assay, 200 μL of 2 mg/mL MTS solution and 10 μL of 0.92 mg/mL phenazine methosulfate (PMS) were added to each well containing 1.0 mL of fresh medium and incubated for 3 h. Absorbance was measured at 490 nm using a microplate reader. For the Live/Dead Assay, the media was aspirated and the samples were stained with 5× Live/Dead dye in PBS. Live (green) and dead (red) cell counts are recorded using a fluorescent microscope.

### PEO Nanofibers:

Open porous networks are essential for cell nutrition, proliferation, and migration for tissue vascularization and formation of new tissues. PEO scaffolds were created with varying fiber diameters to establish a range of pore sizes that facilitate cell infiltration. A single flow pump was used to extrude PEO solution from a 5 ml syringe with an 18-gauge stainless steel needle. The flow rate was varied to create different fiber diameters. Scanning electron microscopy (SEM) was used to observe the morphology of PEO fibers. The fiber diameters were measured from SEM images using ImageJ analysis software. The duration required for the PEO fibers to dissolve in water was recorded as a benchmark for leaching co-electrospun scaffolds.

Co-Electrospinning PCL Collagen Scaffolds with PEO Sacrificial Nanofibers:

Electrospinning parameters established from previous studies were used to co-electrospin PCL/collagen and PEO solutions, simultaneously. Two flow pumps were connected to different positive voltage supplies to extrude PCL/collagen and PEO solutions from separate syringes. Since both needles are connected to positive voltages, the like charges cause repulsion. This can prevent the formation of a continuous scaffold that is composed of both polymers. In such case, the flow pumps are connected to the different positive voltage supplies on opposite sides of the cylindrical drum. PCL/collagen served as the structural component, while the PEO fibers served as the sacrificial component to create porosity. Scaffolds were immersed in water for the duration established in previous studies to leach PEO fibers from the scaffold. The morphology of PCL/collagen fibers was observed with SEM after PEO leaching. In the event of fiber aggregation or the inability of PEO to dissolve, poly(vinyl alcohol) (PVA), a water-soluble polymer, is used as an alternative to create porosity.

### Scaffold Development:

In this study, GSK3β inhibitor-loaded scaffolds were fabricated via electrospinning and hydrogel formation. Pluronic F127 (0.08 g) was dissolved in 1.0 ml hexafluoroisopropanol (HFP) before adding the GSK3β inhibitor. Separately, poly(lactic-co-glycolic acid) PLGA (50:50) (16%, w/v) was dissolved in HFP. The polymer solutions (PLGA/CHIR-F127 or PLGA/CHIR) were electrospun at a flow rate of 1.0 ml/h with a 14 cm distance from the 18G needle tip to the cylindrical drum, and voltages +11/−2 kV were applied. Samples (13.5 mm diameter) punched out from scaffolds were immersed in GelMA (gelatin methacrylate) (5%, w/v)/CHIR solution for 20 seconds, then crosslinked using Lithium Phenyl (2,4,6-Trimethylbenzoyl) Phosphinate (LAP, 0.5% w/v) for 2 minutes on each side under 405 nm UV. Scaffolds comprise a rapidly degrading layer of GelMA for inhibitor delivery to the injury site, and a slow-degrading layer of PLGA to provide prolonged support for tissue regeneration.

### Mechanical Testing:

Uniaxial testing was carried out on PCL/collagen scaffolds in fiber directions using an Instron. A rectangular segment (1 in of width, 10 in of length) from electrospun scaffolds was clamped at its cut ends for axial testing. Segments were placed in PBS before testing. Three regions were tested from each scaffold a total of nine times to compare intra-batch variability. The repeat measurements were averaged to give one value for each scaffold. Additionally, tensile tests were performed on the native supraspinatus muscle and tendons for data comparison.

### Drug Release Assessments:

Samples were submerged in water on a plate shaker at 400 rpm, with 200 μL replaced by fresh water at each time point. The release profile of CHIR99021 was characterized using a UV-vis spectrophotometer at 276 nm and HPLC.

Human skeletal muscle cells (hSkMCs) (5,000 cells/cm²) were cultured in ADM and myogenic differentiation media (myodiff) with scaffolds at 200 rpm, with and without the drug in GelMA and PLGA layers to evaluate the relationship between drug concentrations and lipid formation resulting from the drug's release from the scaffold. PrestoBlue™ assay assessed cell viability.

### Cellular Analysis:

MuSCs and fibroblasts were seeded on scaffolds (10×10 mm²) at a density of 4×10⁴ cells/cm² in their respective growth medium. The location of cells was examined using confocal microscopy after 1, 3, 7, 14, 21, 28, and 62 days of cell-seeding. Confocal imaging enabled the observation of the level of cell infiltration for each scaffold to relate cell infiltration to pore size.

### Statistical Analysis:

For all tests, the average and standard deviation of the data was calculated. All data was evaluated with a One-Way ANOVA with a Tukey's post-hoc test with statistical significance taken as less than 0.05.

### Results

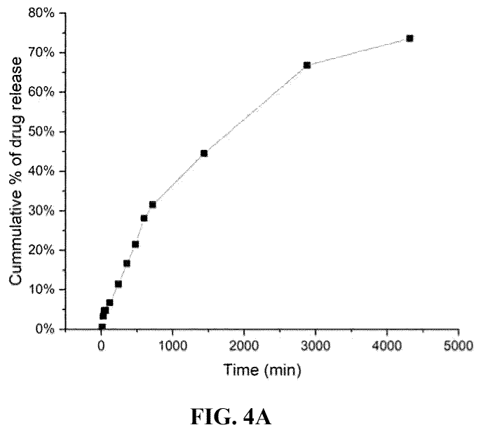

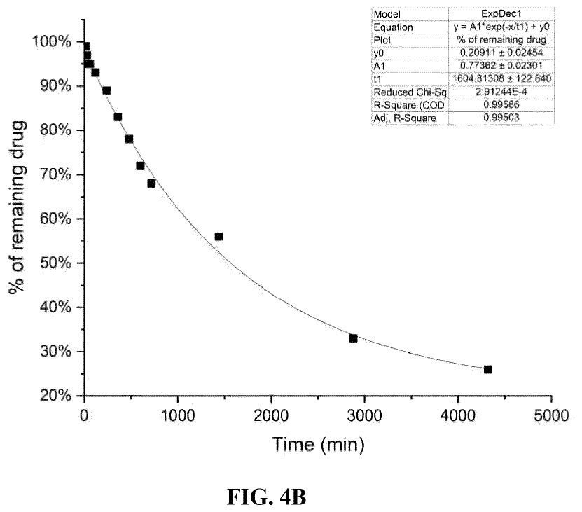

It was determined that CHIR99021 release follows a first-order kinetics profile from polymeric scaffolds with HPLC analysis revealing ˜70% release within 72 hours (FIGS. 4A and 4B). Data from the release studies indicates that suppression of droplet formation is linked to drug concentration in the scaffolds.

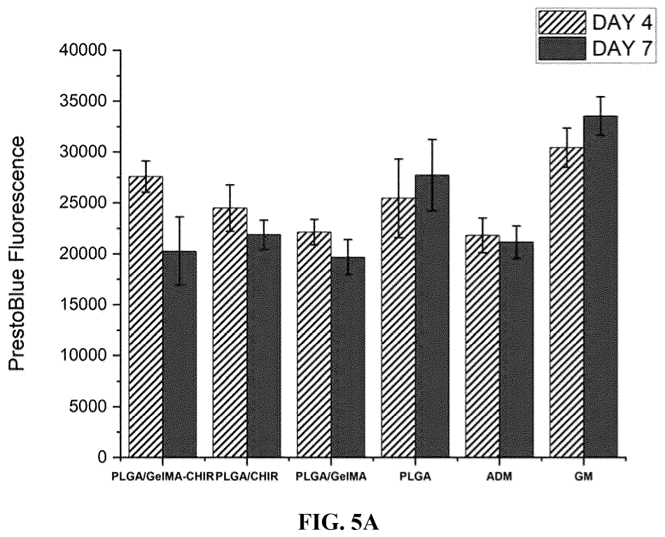

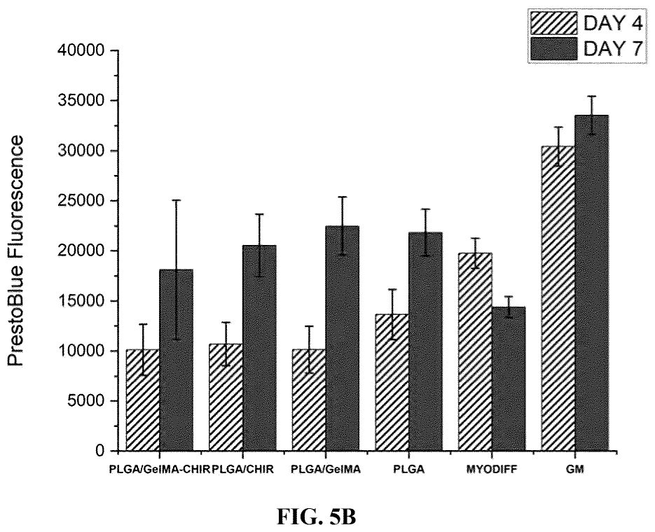

Additionally, morphological assessment of hSkMCs indicated that lipid droplet suppression depends on CHIR99021 concentration within the scaffold. Incorporating CHIR99021 into both GelMA and PLGA layers enhances this effect, highlighting the importance of sustained drug release. Notably, hSkMCs maintained cell viability at day 4 and day 7 in the PLGA/GelMA-CHIR scaffold (FIGS. 5A and 5B).

### Example 3. In Vitro Analysis of GSK-3 Inhibitor in Preventing Adipogenic Differentiation

Electrospun scaffolds are loaded with CHIR99021 (a GSK-3 inhibitor) to evaluate the ability of such inhibitor in preventing fatty infiltration of the MTJ by activating WNT signaling. Cell growth cannot be interrupted by the GSK-3 inhibitor for complete regeneration of MTJ injuries.

### Methods:

### FAP Isolation:

Methods described by Dohmen et al. and Ding et al. are adjusted to isolate FAPs from a rabbit model (Dohmen, R. G. J., et al., Muscle-derived fibro-adipogenic progenitor cells for production of cultured bovine adipose tissue. npj Science of Food, 2022. 6(1): p. 6 and Ding, S., et al., Maintaining bovine satellite cells stemness through p38 pathway. Scientific Reports, 2018. 8(1): p. 10808). FAPs are isolated from the supraspinatus muscle of New Zealand white rabbits. The tissue is minced and dissociated with collagenase 2 and supplemented with 1% PS. 20% FBS in DMEM is also added. after digestion. Muscle fragments are centrifuged, and the supernatant is collected as a cell suspension. Cells are filtered through a 100 m cell strainer and incubated in ammonium-chloride-potassium (ACK) erythrocyte lysis buffer (1 min, room temperature (RT)). Cells are sorted into FAPs by employing a fluorescence-activated cell sorting (FACS) strategy. Prior to FACS, cells are cultured for 72 h on bovine collagen type I coated flasks and sorted using antibodies, CD31 and CD45. CD31−/CD45− cells are sorted into a population of CD29+/CD56− (FAPs) and CD29+/CD56+(MuSCs).

### Assessment of FAP Differentiation:

Adipogenic differentiation of FAPs in response to various concentrations of CHIR99021 is studied. FAPs are seeded at a density of 4×10⁴ cells/cm² in growth medium (F-10 Nutrient mix (Gibco, 31550-023), 20% FBS, 5 ng/mL Recombinant human bFGF, 1% PSA) (Dohmen, R. G. J., et al., Muscle-derived fibro-adipogenic progenitor cells for production of cultured bovine adipose tissue. npj Science of Food, 2022. 6(1): p. 6) and switched to adipogenic differentiation medium (DMEM, 1% FBS, 1% PSA, 1 μM dexamethasone, 10 μg/mL insulin, 5 μM rosiglitazone, 0.5 mM IBMX) (Dammone, G., et al., PPARγ Controls Ectopic Adipogenesis and Cross-Talks with Myogenesis During Skeletal Muscle Regeneration. Int J Mol Sci, 2018. 19(7)) with or without CHIR99021, for 14 days. CHIR99021 is dissolved in DMSO and varied within the range of 0-10 μM (Law, S. M. and J. J. Zheng, Premise and peril of Wnt signaling activation through GSK-3beta inhibition. iScience, 2022. 25(4): p. 104159). Lipid accumulation is assessed with Oil Red O. staining (Abcam: ab287838) following the manufacturer's protocol. Media is removed and cells are fixed with 10% Formalin. Next, the cells are washed with 60% isopropanol, stained with Oil Red O. working solution, and counterstained with Hematoxylin. Lipid droplets, visualized in red, are viewed under microscope. For quantification, Oil Red O. stain is extracted with 100% isopropanol and pipetted into 24-well plates. Absorbance is read at a wavelength of 492 nm using a microplate reader.

Further, an active β-catenin ELISA (Symnansis) is performed following the manufacturer's instructions to assess β-catenin levels in response to CHIR99021. FAPs are grown in media containing CHIR99021. Cell lysate samples are prepared using Cell Lysis Buffer containing 6M Urea. 2×8 antibody-coated microwell strips are placed in a holder and 100 μL of cell lysate is added to wells. After a 2 h incubation period, Biotin-conjugated detection antibody is added for another 2 h incubation at RT. The plate is incubated in the dark for SAV-HRP and TMB substrate solution for 30 min each before the reaction is stopped. Absorbance is read at 450 nm.

### Assessment of MuSC Viability:

MuSCs behavior in response to CHIR99021 is assessed. MuSCs sorted by FACS are plated at 5×10⁴ cells/cm2 on 0.5% Matrigel in growth medium (Ham's F-10 Nutrient Mix, 20% heat-inactivated FBS, 5 ng/mL Recombinant human bFGF, and 1% PSA) for 24 h, and differentiation is initiated by switching to myogenic differentiation medium (DMEM/F-12 Nutrient mix, 2% heat-inactivated FBS, 1% PSA) as described by Dohmen et al. (Dohmen, R. G. J., et al., Muscle-derived fibro-adipogenic progenitor cells for production of cultured bovine adipose tissue. npj Science of Food, 2022. 6(1): p. 6). Cells in media containing CHIR99021 at concentrations obtained from previous studies serve as the experimental samples whereas CHIR99021-free media serve as control samples. Cells are stained with DAPI (nucleus; indicated in blue) and Fluorescein Phalloidin for F-actin (green) to observe myotube formation using a confocal microscope. ImageJ analysis software is used to record the total number of myotubes for each image. The fusion index is calculated by dividing the number of nuclei inside myotubes by the total number of nuclei. RT-qPCR is performed with the primer for α-Actin following the methods from Ding et al. as described below to quantitatively determine the presence of α-Actin (Ding, S., et al., Maintaining bovine satellite cells sternness through p38 pathway. Scientific Reports, 2018. 8(1): p. 10808).

### Assessment of Fibroblast Viability:

Tendon-derived fibroblasts are isolated from the supraspinatus tendon of New Zealand white rabbits following methods from Gögele et al. and Chard et al. (Gögele, C., et al., Cyclically stretched ACL fibroblasts emigrating from spheroids adapt their cytoskeleton and ligament-related expression profile. Cell Tissue Res, 2021. 384(3): p. 675-690 and Chard, M. D. et al. Isolation and growth characteristics of adult human tendon fibroblasts. Ann Rheum Dis, 1987. 46(5): p. 385-90). Fibroblasts are suspended in DMEM containing 10% FBS, 1% PS, and CHIR99021. CHIR99021-free medium serves as a control and CHIR99021 concentration is obtained from previous studies. Live/Dead Assay (ab 115347, Abcam) is performed as previously described to record the effect of CHIR99021 on live cell counts. Fibroblasts must continue proliferating in the presence of CHIR99021 to promote tissue regeneration in vivo. Additionally, CHIR99021 must not interfere with the secretion of ECM proteins such as collagen from fibroblasts. Secreted soluble collagen is detected by taking a sample from culture medium and centrifuging at 1000×g for 15 min at 4° C. to pellet. Then, 20 μL of the clarified supernatant is added to a 96-well plate and the volume is adjusted to 80 uL/well with Collagen Assay Buffer. Collagen concentration is measured using the Collagen Assay Kit (ab241015, Abcam) according to the manufacturer's instructions. The fluorescence is measured at an emission wavelength of 468 nm and an excitation wavelength of 376 nm.

### Scaffold/Fabrication:

The fabrication techniques consist of (1) extruding PCL/collagen from a single syringe and soaking the mat in CHIR99021, (2) spin-coating the mat from (1) with PCL/collagen, (3) extruding PCL/collagen with CHIR99021 from a single syringe, and (4) coaxial electrospinning of PCL in collagen with CHIR99021. PCL and collagen is dissolved in HFP at a total concentration of 5% (wt/vol) and scaffolds are fabricated with a PCL/collagen ratio of 1:1 in weight (Choi, J. S., et al., The influence of electrospun aligned poly(epsilon-caprolactone)/collagen nanofiber meshes on the formation of self-aligned skeletal muscle myotubes. Biomaterials, 2008. 29(19): p. 2899-906). Scaffolds are crosslinked with EDC/NHS following methods from Chen et al. (Chen, D., et al., Electrospun polycaprolactone/collagen nanofibers cross-linked with EDC/NHS and genipin facilitate endothelial cell regeneration and may be a promising candidate for vascular scaffolds.

### CHIR99021 Release Assessment:

The established CHIR99021 concentration must be released from the scaffolds during the initial 3 days of differentiation to inhibit fat accumulation. A UV spectrophotometer is used to detect CHIR99021 release. The scaffolds are immersed in 2 mL of PBS, 1 mL of the supernatant is collected, and the absorbance at a wavelength of 276 nm is measured (Wang, B., et al., A Highly Selective GSK-3β Inhibitor CHIR99021 Promotes Osteogenesis by Activating Canonical and Autophagy-Mediated Wnt Signaling. Front Endocrinol (Lausanne), 2022. 13: p. 926622). CHIR99021 concentration is determined by comparing the absorbance values to a standard curve that relates concentration to absorbance. If the CHIR99021 release cannot be detected, then fabrication techniques are used to create scaffolds with Rhodamine B and measure the release of Rhodamine B. Rhodamine B (479.02 g/mol) and CHIR99021 (465.3 g/mol) have similar molecular weights and are both DMSO soluble. Samples are collected from supernatant containing Rhodamine B and measure fluorescence at an emission wavelength of 568 nm and an excitation wavelength of 546 nm. Rhodamine B concentration is determined by comparing the absorbance values to a standard curve that relates concentration to absorbance.

### Bioactivity Assessment:

It is determined whether the addition of CHIR99021 in PCL/collagen scaffolds affects the activity of CHIR99021. FAPs are seeded on scaffolds with or without CHIR99021, CHIR99021-free scaffolds serving as control samples. Scaffolds are seeded at a density of 4×10⁴ cells/cm² in adipogenic differentiation medium. Oil Red O staining is performed as described by Mauney et al. to assess lipid accumulation (Mauney, J. R. et al. Engineering adipose-like tissue in vitro and in vivo utilizing human bone marrow and adipose-derived mesenchymal stem cells with silk fibroin 3D scaffolds. Biomaterials, 2007. 28(35): p. 5280-90). Following 3 days of cultivation, 4% neutral buffered formalin-fixed samples are cut into 10 μm sections and stained with 60% Oil Red O. solution in PBS. Stain is extracted with 100% isopropanol and the absorbance is measured at a wavelength of 500 nm. Additionally, samples are stained with DAPI (nucleus; indicated in blue), Fluorescein Phalloidin for F-actin (green), and AdipoRed for adipocytes (red) to observe fatty accumulation using a confocal microscope. ImageJ software is used to count lipids.

Second, it is determined whether the addition of CHIR990211 in PCL/collagen scaffolds affects MuSCs and fibroblast growth. This is accomplished by conducting separate cell studies where MuSCs and tendon-derived fibroblasts are seeded on scaffolds with or without CHIR99021. During MuSCs studies, MuSCs is seeded at a density of 5×10⁴ cells/cm² in growth medium for 24 h, and differentiation is initiated by switching to myogenic differentiation medium. After 96 hours, scaffolds are fixed in 4% PFA, permeabilized with 0.5% Triton-X100, and incubated with anti-MHC (1:100) as the primary antibody in 1% BSA. The samples are washed with PBS and incubated with the Goat Anti-Rabbit IgG (H+L) secondary antibody (1:1000, ThermoFisher Scientific). Nuclei are stained with DAPI for confocal imaging. ImageJ analysis software is used to record the total number of myotubes and calculate the fusion index. Next, RT-qPCR is performed following the methods described by Ding et al. to quantitatively determine the presence of myoblast markers including Myosin Heavy Chain (MHC), α-Actin, Myogenin (MYOG) (Ding, S. et al. Maintaining bovine satellite cells stemness through p38 pathway. Scientific Reports, 2018. 8(1): p. 10808). Initial steps include the extraction of RNA from cells and the RNA reverse transcription into cDNA. qPCR is performed using SYBR Green PCR master mix with the primers for MHC, α-Actin, and MYOG. Analysis with qPCR consists of the separation of DNA during denaturation and binding of the primers to complementary parts. Then, primers are extended with DNA polymerase, and two copies of double-stranded DNA form as a result. SYBR green dye binds to double-stranded DNA fragments and fluorescence is monitored after each cycle.

During fibroblast studies, fibroblasts are seeded on scaffolds at a density of 4×10⁴ cells/cm² in DMEM containing 10% FBS and 1% PS. After 7 days of cultivation, scaffolds are fixed in 10% formalin, permeabilized with 0.1% Triton-X, and stained with AlexaFluor 488 Phalloidin (1:1000, ThermoFisher Scientific) and DAPI. Fibroblast morphology is examined using confocal microscopy. RT-qPCR is performed as described to determine collagen type I and elastin expression using primers listed by Kumbar et al. (Kumbar, S. G. et al. Electrospun poly(lactic acid-co-glycolic acid) scaffolds for skin tissue engineering. Biomaterials, 2008. 29(30): p. 4100-7).

### Statistical Analysis:

For all tests, the average and standard deviation of the data for each test is calculated. All data is evaluated with a One-Way ANOVA with a Tukey's post-hoc test with statistical significance taken as less than 0.05.

### Example 4. In Vivo Assessment of PCL/Collagen Co-Electrospun Scaffolds with GSK-3 Inhibitor

This study determines if GSK-3 inhibitor-loaded PCL/collagen scaffolds can reduce the adipogenic differentiation of FAPs while promoting tissue regeneration. Rabbit models replicate clinical characteristics of RC tears, such as muscle retraction, muscle atrophy, fatty infiltration, and inflammation with tenotomy. In this study, skeletally mature female New Zealand white rabbits aged 6 months (N=36) and 5 years (N=36) old are used (Sengupta, P. and S. Dutta, Mapping the Age of Laboratory Rabbit Strains to Human. Int J Prev Med, 2020. 11: p. 194). The age group in rabbits was selected based on the average of 40 years in humans, which is associated with the highest occurrence of myotendinous junction tears as reported by Miranda et al (Miranda, M. O. and N. J. Bureau, Supraspinatus Myotendinous Junction Injuries: MRI Findings and Prevalence. American Journal of Roentgenology, 2018. 212(1): p. W1-W9).

### Methods:

### Surgical Procedure:

The surgical procedure follows the aseptic techniques described by Hyman et al. and Ackerman et al. (Hyman, S. A., et al., In vivo supraspinatus muscle contractility and architecture in rabbit. J Appl Physiol (1985), 2020. 129(6): p. 1405-1412 and Ackerman, J. E. and A. E. Loiselle, Murine Flexor Tendon Injury and Repair Surgery. J Vis Exp, 2016(115)). Rabbits are anesthetized with a subcutaneous injection of ketamine and xylazine (50 and 5 mg/kg) and maintained on 2% isoflurane anesthesia. The surgical site is prepared by clipping the fur on the area of interest and sterilizing the skin by scrubbing with povidone-iodine, followed by 70% ethanol. An incision is made along the scapular spine to expose the superficial shoulder muscles. Rabbit supraspinatus muscle is exposed by dividing the middle trapezius and deltoid muscles to access the vertebral border of the scapula. From the supraspinatus tendon insertion at the acromion and clavicle bones, it is traced 46.7±1.0 mm (Valencia, A. P., et al., A method to test contractility of the supraspinatus muscle in mouse, rat, and rabbit. J Appl Physiol (1985), 2016. 120(3): p. 310-7) up to the myotendinous junction. The tissue is cut at this location. Prior to implantation, scaffolds are sterilized. To minimize the gap between the remaining muscle and tendon, the scaffolds are fitted into the defect horizontally. Additionally, scaffolds with aligned fibers are wrapped and sutured around the site of injury. The electrospun fibers are aligned with the fiber orientation of the muscle and tendon to promote the formation of aligned myotubes and increase the load-bearing ability of the tendon. The control group, consisting of rabbits with complete MTJ tears and no scaffold, undergoes surgical suturing. Lastly, the skin is closed by suturing.

### Assessment of MTJ Tear:

The duration of the study is 9 weeks, in accordance with the lay-off days described by Ekstrand et al. for athletes with large defects at the MTJ (Ekstrand, J. et al. Return to play after thigh muscle injury in elite football players: implementation and validation of the Munich muscle injury classification. Br J Sports Med, 2013. 47(12): p. 769-74). Each assessment is performed with 6 rabbits from each of the control and experimental (6-month and 5-year-old) groups. Rabbits are sacrificed at 0 (n=6, only control) and 3 (n=18) postoperative days for initial assessments as fat accumulation likely occurs in the first 3 days of injury. Additionally, rabbits are sacrificed at 1 (n=18), 2 (n=18), 4(n=18), 7(n=18), or 9 (n=18) postoperative weeks where the shoulder is dissected to harvest supraspinatus MTJ. Injury site is observed through MRI. MRI T2 sequences detect muscle injury by the presence of hyperintense fluid accumulation, while T1 sequences detect fatty muscular infiltration (Nescolarde, L. et al. Differentiation Between Tendinous, Myotendinous and Myofascial Injuries by L-BIA in Professional Football Players. Front Physiol, 2020. 11: p. 574124).

The sections are stained with hematoxylin and eosin (H&E) to qualitatively assess cellular infiltration and tissue development. Masson's trichrome and Oil Red O. stains are also applied to visualize collagen and fat content. Oil Red O. staining is performed following the methods described by Kuwahara et al. (Kuwahara, Y. et al. Fatty degeneration and wnt10b expression in the supraspinatus muscle after surgical repair of torn rotator cuff tendon. J Orthop Surg (Hong Kong), 2019. 27(3): p. 2309499019864817). Briefly, tissues are frozen in isopentane and cooled with liquid nitrogen. Sections of 5 μm are prepared using a cryostat and stained with Oil Red O solution for 30 min and counter-stained with hematoxylin. A cylinder cut from a 12 mm diameter polypropylene tube is placed on the slides surrounding the stained section and fixed in an aqueous mounting medium. The stain is extracted from three sections with 100 μL of 100% isopropyl alcohol and the absorbance is measured by a spectrophotometer at 510 nm. Masson's trichrome (VitroVivo Biotech) is performed with frozen tissue sections fixed in 10% formalin according to the manufacturer's instructions. Sections are placed in Bouin's reagent at 60° C. for 1 hr, counterstained with Weigert's working hematoxylin for 7 min, and placed in Biebrich solution for 5 min, followed by rinsing in distilled water for 5 min after each immersion. Afterwards, Phosphotungstic/phosphomolybdic Acid, and Aniline Blue solutions are applied each for 5 min. Sections are dehydrated with 70% ethanol. As a result, collagen sections are stained blue, while muscle fibers are stained red.

What is claimed is:

1. An electrospun polymer fiber scaffold comprising a biocompatible and bioresorbable polymer fibers that are blended with an amount of a GSK-3 inhibitor.

2. The scaffold of claim 1, wherein the polymer is selected from the group consisting of poly(lactic-co-glycolic acid) (PLGA), poly-(glycolic acid) (PGA), polycaprolactone (PCL), polyesteramide (PEA), polyphosphazene, and poly(L-lactic acid) (PLA).

3. The scaffold of claim 2, wherein the polymer is soaked in or coated with gelatin.

4. The scaffold of claim 3, wherein the gelatin comprises cross-linked gelatin methacrylate.

5. The scaffold of claim 2, wherein the scaffold comprises collagen fibers.

6. The scaffold of claim 5, wherein the PCL to collagen ratio ranges from about 1:1 to about 4:1.

7. The scaffold of claim 1, wherein the amount of the GSK-3 inhibitor comprises 0.5 μM to 2.0 μM.

8. The scaffold of claim 1, wherein the scaffold comprises pores of about 200 μm to about 500 μm in diameter.

9. The scaffold of claim 1, wherein the GSK-3 inhibitor is CHIR99021.

10. A method of repairing a composite tissue injury susceptible to adipocyte infiltration, comprising applying to the injury an electrospun polymer fiber scaffold comprising biocompatible and bioresorbable polymer fibers that are blended with an amount of a GSK-3 inhibitor that is effective to inhibit the infiltration of adipocytes into the scaffold and tissue.

11. The method of claim 12, wherein the polymer is soaked in or coated with gelatin.

12. The method of claim 13, wherein the gelatin comprises cross-linked gelatin methacrylate.

13. The method of claim 12, wherein the composite tissue injury susceptible to adipocyte infiltration is a myotendinous junction (MTJ) injury.

14. The method of claim 17, wherein the MTJ injury is a rotator cuff medial tear.

15. The method of claim 18, wherein the tear is a type A tear, a type B tear, or a type C tear.

16. A kit comprising the electrospun polymer fiber scaffold of claim 1.

17. The kit of claim 16, further comprising muscle stem cells (MuSCs).

18. A method of preventing infiltration of adipocytes into a scaffold and tissue following a composite tissue injury, comprising administering to a subject in need thereof an electrospun polymer fiber scaffold comprising biocompatible and bioresorbable polymer fibers that are blended with a GSK-3 inhibitor that is effective to inhibit the infiltration of adipocytes into the scaffold and tissue.

19. The method of claim 18, wherein the polymer is selected from the group consisting of poly(lactic-co-glycolic acid) (PLGA), poly-(glycolic acid) (PGA), polycaprolactone (PCL), polyesteramide (PEA), polyphosphazene, and poly(L-lactic acid) (PLA).

20. The method of claim 18, wherein the composite tissue injury is a MTJ injury.
